(19) United States
(12) Patent Application Publication
(10) Pub. No.: US 2025/0383330 A1
(43) Pub. Date: Dec. 18, 2025

(54) COLORIMETRIC DETECTION SYSTEM FOR DETECTING CHEMICAL COMPOUNDS USING A COLOR INDICATOR OF THE HYDRAZONE FAMILY

(71) Applicant: COMMISSARIAT A L'ENERGIE ATOMIQUE ET AUX ENERGIES ALTERNATIVES, Paris (FR)

(72) Inventors: Lucile TERMEAU, Grenoble Cedex 09 (FR); Sébastien PENLOU, Grenoble Cedex 09 (FR); Alexandre CARELLA, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 18/718,448

(22) PCT Filed: Dec. 15, 2022
(86) PCT No.: PCT/FR2022/052383

§ 371 Date: Jun. 11, 2024

(30) Foreign Application Priority Data
Dec. 16, 2021 (FR) FR2113669

(51) Int. Cl. G01N 31/22; G01N 21/78

(52) U.S. Cl. CPC G01N 31/22; G01N 21/78

(57) ABSTRACT

A colorimetric detection system may be use to detect a chemical compound, and may include a support including a color indicator for the chemical compound to be detected. The color indicator may be a conjugated hydrazone compound. The conjugated hydrazone compound may include (i) at least one cyclic group bonded to the carbon atom of the hydrazone function and (ii) an aromatic group bonded to nitrogen atom located in a β-position to the carbon atom of the hydrazone group, the aromatic group including at least one electron-withdrawing group bonded to the nitrogen atom of the hydrazone function

### TECHNICAL FIELD

The present invention relates to a novel colorimetric detection system usable for the detection of chemical compounds, in particular, toxic warfare compounds, using a color indicator of the hydrazone family and to a method for detecting the presence or the absence of chemical compounds implementing this colorimetric detection system.

In particular, the present invention may apply to the detection of toxic chemical compounds, such as toxic warfare compounds and, in particular, organophosphorus compounds; toxic industrial chemical compounds (known under the name TIC), pesticides.

Generally, organophosphorus compounds are in the form of organic compounds having a proven toxicity for the human organism. Indeed, these compounds may be involved in the mechanism of inhibiting serine proteases and, in particular, acetylcholinesterase, which intervenes in the synaptic junctions and of which the dysregulationof the activity may prevent muscular relaxation and thus cause death by asphyxia.

These compounds may be included in the formulation of insecticides, pesticides or also chemical warfare agents (such as G-serie organophosphorus compounds, such as sarin (GB, CAS no. 107-44-8) or V-serie organophosphorus compounds, such as VX (CAS no. 50782-69-9) and due to the high lethality of these compounds, their proliferation, it is important to be able to have preliminary detection and identification systems.

Some detection systems used to date are based on technologies involving physical measurement means, such as ion mobility spectroscopy, flame photometry, IR and Raman spectroscopies with, for difficulties, that these systems require complex expensive equipment and are not necessarily suitable for all working environments in terms of mass and size, to which the expertise of the operator to be considered is added.

In view of the existing, the authors of the present invention focused on the design of a detection system usable for the detection of chemical compounds, such as organophosphorus compounds, from a specific color indicator.

### DESCRIPTION OF THE INVENTION

Thus, the invention relates to a colorimetric detection system usable for the detection of a chemical compound, comprising a support comprising a color indicator of said chemical compound to be detected, characterised in that the color indicator is a conjugated hydrazone compound.

Color indicator (also known as colorimetric detection indicator) means, conventionally, a chemical substance that takes at least one characteristic color in the presence of a chemical compound namely, in other terms, a chemical substance that has at least two colored states, one existing colored state when the chemical substance is not in the presence of the chemical compound to be detected and at least one other colored state when the chemical substance is in the presence of the chemical compound to be detected.

The constituent support of the detection system may be, in particular, a support comprising fibreglass or is a paper support (for example, chromatography paper), with a preference for the support comprising fibreglass, or is a support comprising a powder (for example, a silica powder, a polyethylene powder), being understood that this support must be capable of being impregnated with the conjugated hydrazone compound.

Conjugated hydrazone means a compound comprising a hydrazone function of formula —NH—N═C—, of which the double bond is conjugated with another double bond, namely, in other terms, that the carbon atom bearing the double bond of the hydrazone function is bonded to another carbon atom bearing a double bond, which may be schematically represented by the following formula (I):

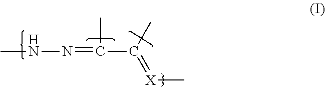

X indicating a carbon atom or a heteroatom, the braces indicating that the atoms are bonded to other atoms to reach their valence, this formula also covering the tautomer forms. Further, when the nitrogen atom of the hydrazone function will be mentioned, it means the nitrogen atom not bearing the double bond, this nitrogen atom being that represented to the left of the formula (I). It is also understood that when X is a carbon atom, it will include an additional valence relative to that which is represented on the formula (I).

More specifically, the other carbon bearing a double bond may belong to an aromatic, possibly heteroaromatic, group or may belong to an ethylenic group.

According to a first embodiment, the conjugated hydrazone compound comprises at least one cyclic group bonded to the carbon atom bearing the double bond of the hydrazone function and further comprises an aromatic group bearing at least one electron-withdrawing group (such as NO₂), bonded to the nitrogen atom of the hydrazone function.

Hydrazone compounds complying with the specific features of this first embodiment may be compounds of which the cyclic group(s) bonded to the carbon atom bearing the double bond of the hydrazone function is (are) carbon cyclic group(s) (that is to say of which the atoms of the cycle are all carbon atoms, which does not exclude that these carbon atoms may be bonded to groups bearing one or more heteroatoms), for example carbon aromatic groups and, also more specifically, hydrazone compounds of which the cyclic group(s) bonded to the carbon atom bearing the double bond of the hydrazone function is (are) carbon cyclic group(s) (for example carbon aromatic groups) and the aromatic group bearing at least one electron-withdrawing group is an aromatic group bearing at least one NO₂ group.

Hydrazone compounds complying with these criteria, can comply with one of the following formulae (II), (III), (IV), (V), (VI) and (VI′):

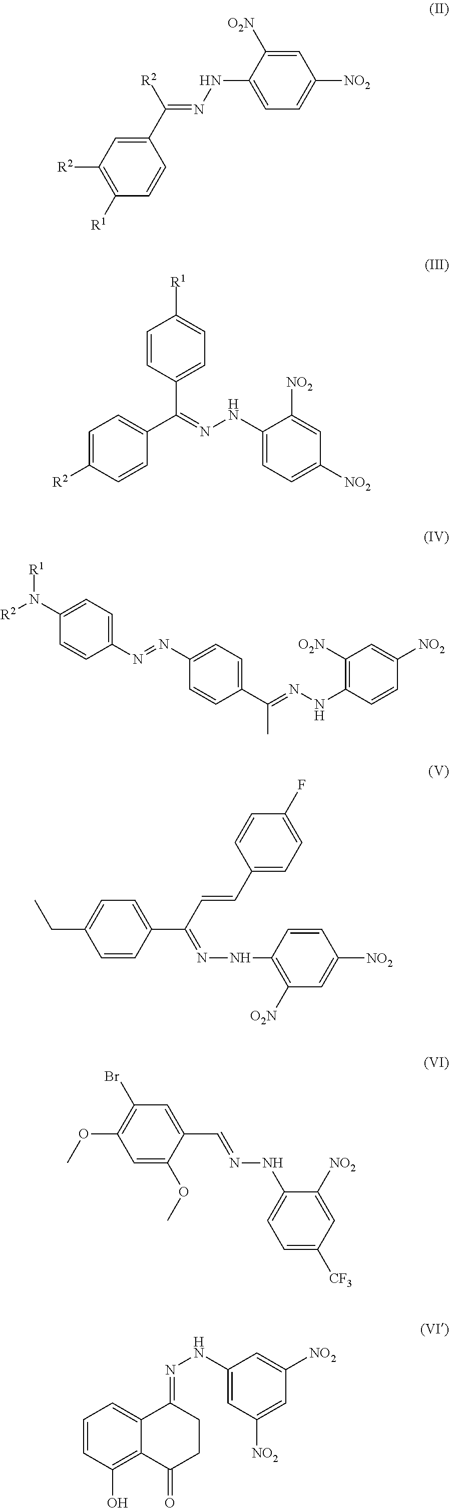

wherein:
   for the formula (II), R¹ represents —N(CH₃)₂, —CH₃, —OCH₃, —OH, —SCH₃ or —NO₂;
   R² represents —H or —OCH₃; R³ represents —H or —CH₃;
   for the formula (III), R¹ and R² represent, independently of one another, —H, —N(CH₃)₂, —OCH₃ or —NO₂;
   for the formula (IV), R¹ and R² represent, independently of one another, —CH₃ or —CH₂—CH₃.

More specifically, particular compounds entering in the definition of the compounds of formula (II) are the following:
the compound wherein R¹ represents —N(CH₃)₂, R² represents —H and R³ represents H (compound hereinafter referenced as “compound 1a”);
the compound wherein R¹ represents —CH₃, R² represents H and R³ represents H (compound hereinafter referenced as “compound 1b”);
the compound wherein R¹ represents —OCH₃, R² represents —H and R³ represents H (compound hereinafter referenced as “compound 1c”);
the compound wherein R¹ represents —OH, R² represents —OCH₃ and R³ represents H (compound hereinafter referenced as “compound 1d”);
the compound wherein R¹ represents —SCH₃, R² represents —H and R³ represents H (compound hereinafter referenced as “compound 1e”);
the compound wherein R¹ represents —NO₂, R² represents —H and R³ represents —CH₃ (compound hereinafter referenced as “compound 1f”).

More specifically, particular compounds entering in the definition of the compounds of formula (III) are the following:
the compound wherein R¹ represents —H and R² represents —H (compound hereinafter referenced as “compound 3a”);
the compound wherein R¹ represents —N(CH₃)₂, R² represents —N(CH₃)₂ (compound hereinafter referenced as “compound 3b”); and
the compound wherein R¹ represents —OCH₃ and R² represents —OCH₃ (compound hereinafter referenced as “compound 3c”).

More specifically, particular compounds entering in the definition of the compounds of formula (IV) are the following:
the compound wherein R¹ represents —CH₃ and R² represents —CH₃ (compound hereinafter referenced as “compound 5a”); and
the compound wherein R¹ represents —CH₂—CH₃, R² represents —CH₂—CH₃ (compound hereinafter referenced as “compound 5b”).

Hydrazone compounds complying with the specific features of the first embodiment may also be compounds of which the cyclic group(s) bonded to the carbon atom bearing the double bond of the hydrazone function is (are) heteroaromatic group(s) (that is to say a group of which at least one atom of the cycle(s) is a heteroatom, such as O, N, S) and, more specifically, hydrazone compounds of which the cyclic group(s) bonded to the carbon atom bearing the double bond of the hydrazone function is (are) heteroaromatic group(s) and the aromatic group bearing at least one electron-withdrawing group is an aromatic group bearing at least one NO₂ group.

Hydrazone compounds complying with these criteria, can comply with one of the following formulae (VII), (VIII), (IX), (X) and (XI):

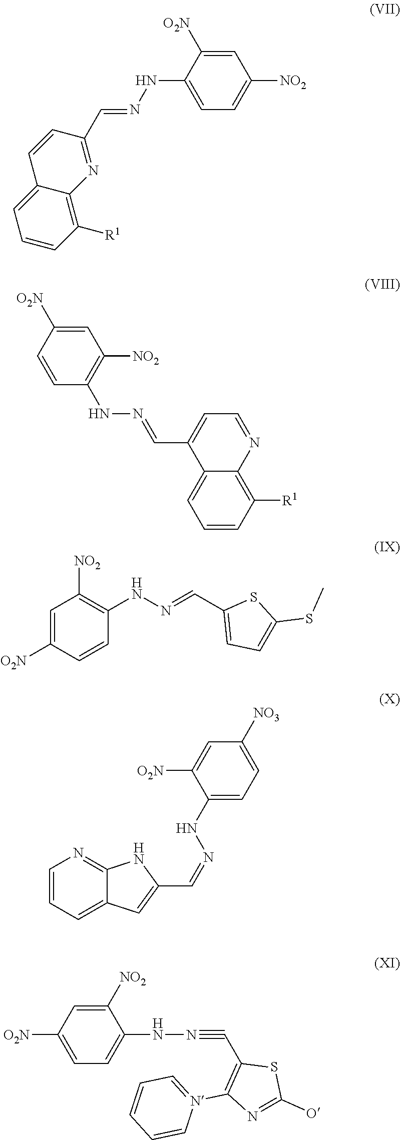

wherein:
   for the formula (VII), R¹ represents —H or —OH;
   for the formula (VIII), R¹ represents —H or —OH.

More specifically, particular compounds entering in the definition of the compounds of formula (VII) or (VIII) are the following:
the compound wherein R¹ represents —H (compound hereinafter referenced as “compound 2a”):
the compound wherein R¹ represents —OH (compound hereinafter referenced as “compound 2b”).

According to a second embodiment, the conjugated hydrazone compound comprises at least one ethylenic group bonded to the carbon atom bearing the double bond of the hydrazone function and further comprising an aromatic group bearing at least one electron-withdrawing group (such as NO₂ or a heteroaromatic group) bonded to the nitrogen atom of the hydrazone function, particular compounds complying with this specific feature, complying with one of the following formulae (XII) or (XIII):

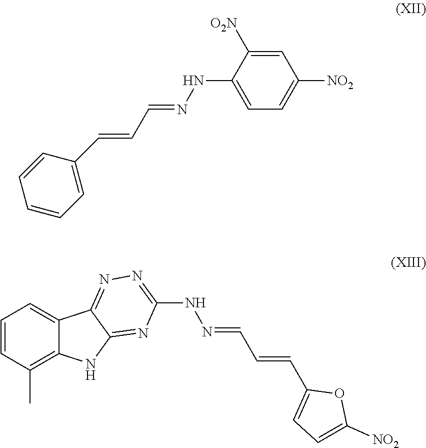 [SMILES: O=[N+]([O-])c1ccc(N/N=C/C=C/c2ccccc2)c([N+](=O)[O-])c1 ; Cc1cccc2c1[nH]c1nc(N/N=C/C=C/c3ccc([N+](=O)[O-])o3)nnc12]

According to a third embodiment, the conjugated hydrazone compound comprises at least one aromatic group bearing an —OH group bonded to the nitrogen atom of the hydrazone function or to the carbon atom bearing the double bond of the hydrazone function, particular compounds complying with this specific feature, complying with one of the following formulae (XIV), (XV), (XVI), (XVII), (XVIII), (XIX) and (XX):

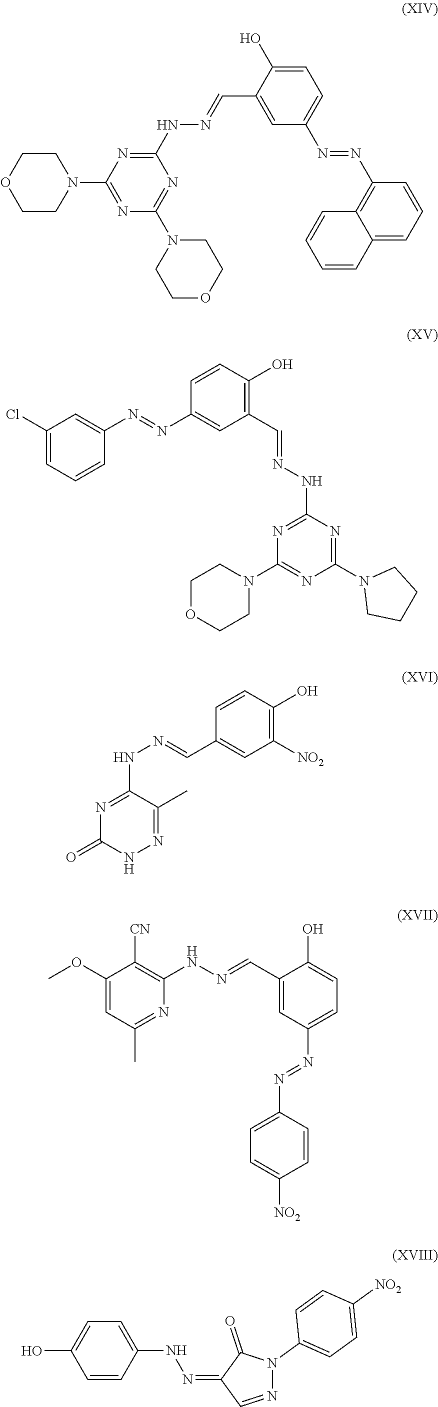 [SMILES: Oc1ccc(/N=N/c2cccc3ccccc23)cc1/C=N/Nc1nc(N2CCOCC2)nc(N2CCOCC2)n1 ; Oc1ccc(/N=N/c2cccc(Cl)c2)cc1/C=N/Nc1nc(N2CCCC2)nc(N2CCOCC2)n1 ; Cc1n[nH]c(=O)nc1N/N=C/c1ccc(O)c([N+](=O)[O-])c1 ; COc1cc(C)nc(N/N=C/c2cc(/N=N/c3ccc([N+](=O)[O-])cc3)ccc2O)c1C#N ; O=C1/C(=N\Nc2ccc(O)cc2)C=NN1c1ccc([N+](=O)[O-])cc1]

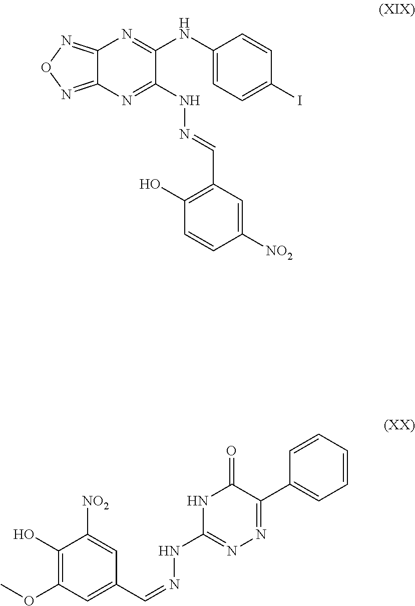 [SMILES: O=[N+]([O-])c1ccc(O)c(/C=N/Nc2nc3nonc3nc2Nc2ccc(I)cc2)c1 ; COc1cc(/C=N\Nc2nnc(-c3ccccc3)c(=O)[nH]2)cc([N+](=O)[O-])c1O]

According to a fourth embodiment, the conjugated hydrazone compound comprises a heteroaromatic group bonded, possibly via a spacer group (such as a —CO— group), to the nitrogen atom of the hydrazone function and another heteroaromatic group bonded to the carbon atom bearing the double bond of the hydrazone function or comprises a dizoaromatic group bonded to the nitrogen of nitrogen of the hydrazone function and a heteroaromatic group encompassing the carbon atom bearing the double bond of the hydrazone function, particular compounds complying with this specific feature, complying with one of the following formulas (XXI), (XXII), (XXIII) and (XXIV):

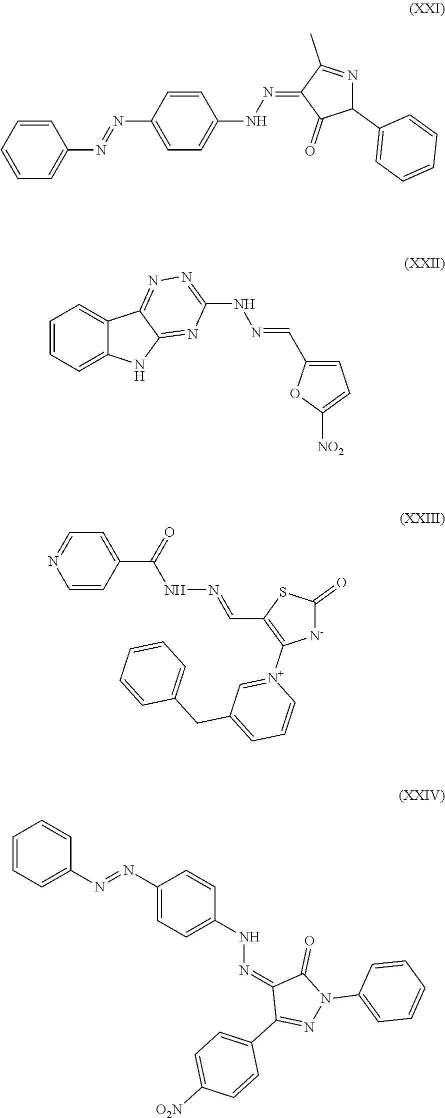 [SMILES: CC1=NC(c2ccccc2)C(=O)/C1=N\Nc1ccc(/N=N/c2ccccc2)cc1 ; O=[N+]([O-])c1ccc(/C=N/Nc2nnc3c(n2)[nH]c2ccccc23)o1 ; O=C(N/N=C/c1sc(=O)[n-]c1-[n+]1cccc(Cc2ccccc2)c1)c1ccncc1 ; O=C1/C(=N\Nc2ccc(/N=N/c3ccccc3)cc2)C(c2ccc([N+](=O)[O-])cc2)=NN1c1ccccc1]

From the abovementioned hydrazone compounds, some are novel and are the object of the invention, these compounds complying with one of the following formulae (5a), (5b) and (X):

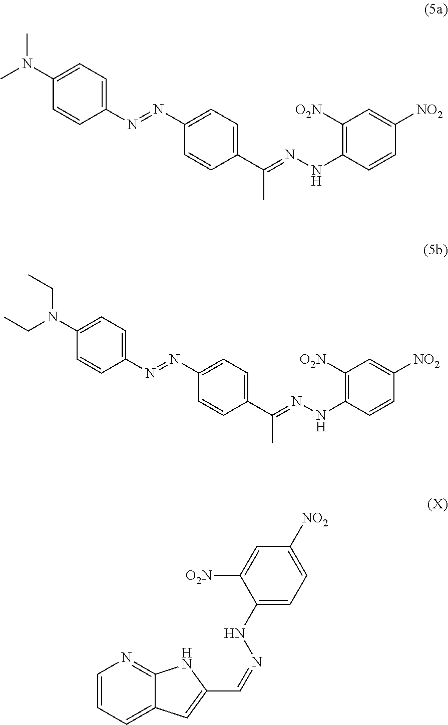 [SMILES: C/C(=N\Nc1ccc([N+](=O)[O-])cc1[N+](=O)[O-])c1ccc(/N=N/c2ccc(N(C)C)cc2)cc1 ; CCN(CC)c1ccc(/N=N/c2ccc(/C(C)=N/Nc3ccc([N+](=O)[O-])cc3[N+](=O)[O-])cc2)cc1 ; O=[N+]([O-])c1ccc(N/N=C\c2cc3cccnc3[nH]2)c([N+](=O)[O-])c1]

These compounds may be obtained conventionally by a condensation reaction between a carbonyl compound (for example, a ketone or an aldehyde) and a compound comprising a hydrazine function.

The detection systems in accordance with the invention may be prepared by a method comprising a step of depositing the color indicator on the support by projecting an ink comprising the color indicator thereon.

Regarding the possible deposition techniques, this may be a deposition with the micropipette, a printing (for example, via a Dimatix printer), a deposition via particles impregnated by the appropriate color indicator or a deposition by screen printing.

Finally, the invention also relates to a method for detecting the presence or the absence of a chemical compound comprising the following steps:
a step of placing in contact the medium or media, of which it is desired to detect the presence or the absence of said chemical compound with the detection system as defined above;
a step of deducing, depending on the possible chromatic changes observed, the presence or the absence of said chemical compound.

This step of placing in contact may consist in depositing at the surface of the detection system one or more distinct drops of each medium of which it is desired to analyse the absence or the presence of a chemical compound.

It is understood that the colorimetric detection system that must be used within the scope of the abovementioned method, must be capable of detecting the chemical compound, of which it is desired to determine the absence or the presence.

Between the step of placing in contact and the step of deducing, a waiting time may be provided so that, if applicable, the chromatic change can take place.

Regarding the step of deducing, the operator may base themselves on a colorimetric scale associated with the detection system, which will define, for all of the chemical compounds likely to be detected by the system, the corresponding chromatic change, this colorimetric scale being able to be determined, by preliminary tests, for each of the systems and the chemical compounds intended to be detected by said systems. The deducing may be carried out with the naked eye or, if necessary, via opto-electronic means.

The compounds likely to be detected by the method of the invention may be toxic warfare compounds; toxic industrial compounds, pesticides and more specifically, may be organophosphorus compounds and, also more specifically, toxic organophosphorus compounds complying with one of the following formulae (XXV) and (XXVI):

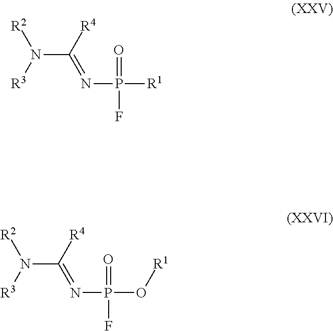

wherein:
   R¹ represents H or an alkyl group, possibly cyclic, that can comprise up to 10 carbon atoms;
   R² and R³ represent, independently of one another, an alkyl group, possibly cyclic that can comprise up to 10 carbon atoms;
   R⁴ represents H, an alkyl group, possibly cyclic, that can comprise up to 10 carbon atoms or an amino group.

In particular, the detection systems, particularly when the support is made of fibreglass and the color indicator is a hydrazone compound 1b, 1d, 1f, 2a, 2b or 3c as defined above or a compound of formula (IX) as defined above, are particularly adapted to the selective detection of toxic organophosphorus compounds complying with one of the following formulae (XXV) and (XXVI):

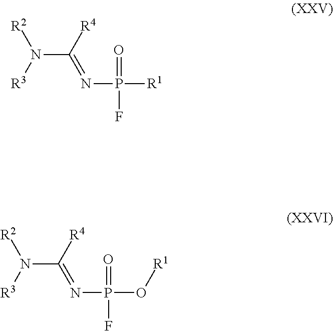

wherein:
   R¹ represents H or an alkyl group, possibly cyclic that can comprise up to 10 carbon atoms;
   R² and R³ represent, independently of one another, an alkyl group, possibly cyclic that can comprise up to 10 carbon atoms;
   R⁴ represents H, an alkyl group, possibly cyclic, that can comprise up to 10 carbon atoms or an amino group.

Specific organophosphorus compounds entering in this category are the specific compounds complying with one of the following formulae (XXVII), (XXVIII), (XXIX) and (XXX):

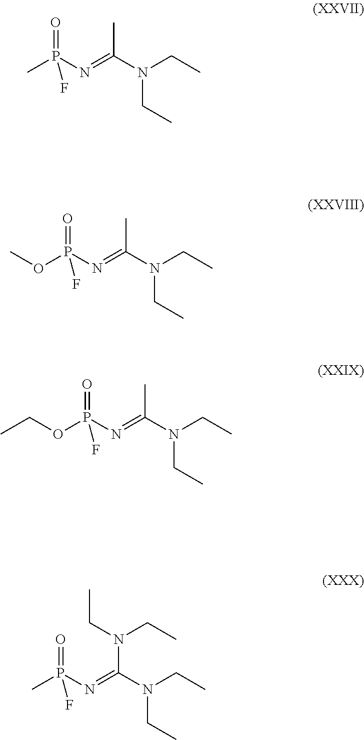

Finally, the invention also relates to a colorimetric detection kit usable for the detection of a chemical compound comprising the following elements:
a detection system in accordance with the invention and as defined above; and
a colorimetric scale for making the correspondence between the chromatic change observed and the chemical compound detected.

Other features and advantages of the invention will become more apparent upon reading the following additional description, which relates to an example of preparing a detection system in accordance with the invention.

Of course, the following example is only given by way of illustration of the subject matter of the invention and, under no circumstances, constitutes a limitation of this subject matter.

### DETAILED DESCRIPTION OF PARTICULAR EMBODIMENTS

### Example 1

This example illustrates the preparation of various specific conjugated hydrazone compounds usable as color indicator within the scope of the invention, this preparation being performed by condensation of the hydrazine function borne by 2,4-dinitrophenylhydrazine with the carbonyl function —C═O borne by a carbonyl reactant.

The general protocol for preparing these hydrazone compounds is the following.

First, 2,4-dinitrophenylhydrazine (1.3 eq.) is solubilised with heat (50° C.) at 0.1 M in ethanol, in the presence of concentrated sulphuric acid (3 eq.). An ethanolic solution containing the carbonyl reactant (1 eq.) is subsequently added drop by drop into the solution comprising 2,4-dinitrophenylhydrazine. The reaction mixture is subsequently refluxed until complete conversion of the reactants. In the case of aldehyde-derived reactants, the reaction is almost instantaneous and adding at a temperature of 50° C. is sufficient. The hydrazone formed is only partially soluble in ethanol and it forms product suspensions in the reaction medium at the end of the reaction. After cooling at room temperature, the reaction medium is evaporated at 3/4. The residue is subsequently partially dissolved in a volume of deionised water and treated by a basic solution of NaOH at 1 M. A precipitation of the hydrazone occurs that is then filtered and rinsed with deionised water at least twice. The crude solid is subsequently hot-washed in the suitable solvent, generally in ethanol. It is subsequently dried in the oven (80° C.) then under vane pump vacuum.

Some modalities have been carried out depending on the carbonyl derivatives selected and are described more precisely for each of the hydrazones.

### a) Precise Modalities for Preparing Compound 1a

The paragraph below illustrates the precise modalities for preparing compound 1a complying with the following formula:

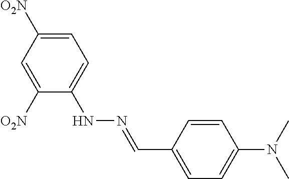 [SMILES: CN(C)c1ccc(/C=N/Nc2ccc([N+](=O)[O-])cc2[N+](=O)[O-])cc1]

2,4-dinitrophenylhydrazine (estimated purity at 68%, 4.74 mmol, 1.382 g, 1.3 eq.) is reacted according to the general synthetic protocol with 4-dimethylaminobenzaldehyde (3.64 mmol, 549.1 mg, 1 eq.). The reaction mixture is stirred at 50° C. for 15 min. The hydrazone compound (1a) is obtained in the form of a black color powder (0.98 g, 98%).

The IR and ¹H RMN spectroscopic results figure below.

IR: 3,302 (NH), 3,135 to 2979 (Ar—H), 1,604 (C═N), 1,583, 1,406, 1,333, 1,281, 1,125, 1,062, and 698 cm−1;

¹H RMN (DMSO-d₆, 400 MHz): δ 11.57 (s, 1H, N—H), 8.872 (s, 1H, ArDNPH-H), 8.553 (s, 1H, ArDNPH-H), 8.346 (d, 1H, ArDNPH-H), 8.045 (d, 1H, N═CH), 7.623 (d, 2H, Arcarbonyl-H), 6.787 (d, 2H, Arcarbonyl-H), 3.010 (s, 6H, N—CH₃).

### b) Precise Modalities for Preparing Compound 1b

The paragraph below illustrates the precise modalities for preparing compound 1b complying with the following formula:

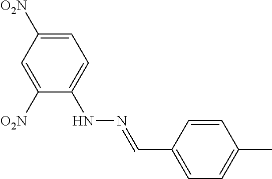 [SMILES: Cc1ccc(/C=N/Nc2ccc([N+](=O)[O-])cc2[N+](=O)[O-])cc1]

P-tolualdehyde (0.333 mmol, 0.04 mL, 1 eq.) in liquid form is directly added while hot on 2,4-dinitrophenylhydrazine (0.43 mmol, 126.3 mg, 1,3 eq.). The reaction mixture is stirred at 50° C. for 15 min. The hydrazone compound (1b) is obtained in the form of an orange color powder (86.9 mg, 86.9%).

The ¹H RMN spectroscopic results figure below.

¹H RMN (CDCl₃, 400 MHz): δ 11.57 (s, 1H, N—H), 9.149 (d, 1H, ArDNPH-H), 8.35 (dd, 1H, ArDNPH-H), 8.08 (s, 1H, ArDNPH-H), 8.04 (s, 1H, N═CH), 7.667 (d, 2H, Arcarbonyl-H), 7.282 (s, IH, Ar—H), 2.422 (s, 3H, Ar—CH₃).

### c) Precise Modalities for Preparing Compound 1c

The paragraph below illustrates the precise modalities for preparing compound 1c complying with the following formula:

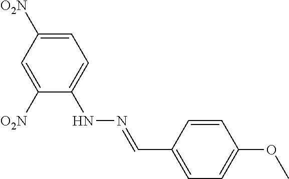 [SMILES: COc1ccc(/C=N/Nc2ccc([N+](=O)[O-])cc2[N+](=O)[O-])cc1]

Anisaldehyde (0.32 mmol, 0.038 mL, 1 eq.) in liquid form is directly added while hot on 2,4-dinitrophenylhydrazine (0.41 mmol, 119.9 mg, 1.3 eq.). The reaction mixture is stirred at 50° C. for 15 min. The hydrazone compound (1c) is obtained in the form of an orange-red color powder (73.4 mg, 73.4%).

The ¹H RMN spectroscopic results figure below.

¹H RMN (CDCl₃, 400 MHz): δ 11.57 (s, 1H, N—H), 9.151 (d, 1H, ArDN PH—H), 8.342 (dd, 1H, ArDNPH-H), 8.0615 (s, 1H, ArDNPH-H), 8.04 (s, 1H, N═CH), 7.724 (d, 2H, Arcarbonyl-H), 6.984 (d, 2H, Arcarbonyl-H), 3.882 (s, 3H, Ar—OCH₃)

### d) Precise Modalities for Preparing Compound 1d

The paragraph below illustrates the precise modalities for preparing compound 1d complying with the following formula:

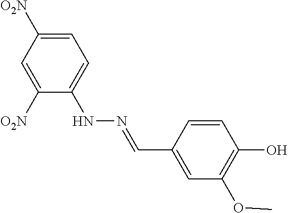 [SMILES: COc1cc(/C=N/Nc2ccc([N+](=O)[O-])cc2[N+](=O)[O-])ccc1O]

2,4-dinitrophenylhydrazine (0.452 mmol, 131.8 mg, 1 eq.) is reacted according to the general synthetic protocol with vanilline (0.452 mmol, 69.4 mg, 1 eq.). The reaction mixture is stirred at 50° C. for 1 h. In the case of this hydrazone, no basic treatment is performed. The product is only rinsed with ethanol. The hydrazone compound (1d) is obtained in the form of a red powder (146.5 mg, 97.5%) The IR and ¹H RMN spectroscopic results figure below.

IR (cm−1) v: 3,385 (O—H), 3,278 (NH), 1,622 (C═N), 1,515 (NO2), 1,418, 1,334, 1,268 (C-0), 1,134, 1,062 and 865

¹H RMN (DMSO-d₆, 500 Mhz) δ (ppm): 11.59 (s, 1H, N—H), 9.694 (s, 1H, OH), 8.88 (s, 1H), 8.584 (s, 1H, N═CH), 8.35 (d, 1H, Ar—H), 8.10 (d, J=9.66 Hz, 1H), 7.40 (s, 1H), 7.18 (d, 1H), 6.88 (d, J=8.1 Hz, 1H), 3.875 (s, 3H, OCH 3)

### e) Precise Modalities for Preparing Compound 1e

The paragraph below illustrates the precise modalities for preparing compound 1e complying with the following formula:

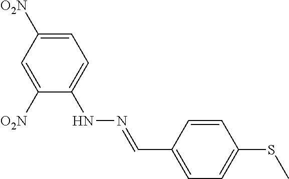 [SMILES: CSc1ccc(/C=N/Nc2ccc([N+](=O)[O-])cc2[N+](=O)[O-])cc1]

4-methylthio benzaldehyde (0.602 mmol, 82.51 μL, 1 eq.) in liquid form is directly added while hot on 2,4-dinitrophenylhydrazine (0.783 mmol, 0.228 g, 1,3 eq.). The reaction mixture is stirred at 50° C. for 2 h. The hydrazone compound (1e) is obtained in the form of an orange-red color powder (168 mg, 84%).

The ¹H RMN spectroscopic results figure below.

¹H RMN (CDCl₃, 500 MHz): δ 11.306 (s, 1H, N—H), 9.155 (s, 1H), 8.353 (ddd, 1H), 8.080 (t, 2H), 7.682 (d, 2H), 7.30 (d, 2H), 3.541 (s, 3H)

### f) Precise Modalities for Preparing Compound 1f

The paragraph below illustrates the precise modalities for preparing compound 1f complying with the following formula:

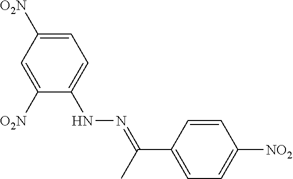 [SMILES: C/C(=N\Nc1ccc([N+](=O)[O-])cc1[N+](=O)[O-])c1ccc([N+](=O)[O-])cc1]

2,4-dinitrophenylhydrazine (0.75 mmol, 219.7 mg, 1.3 eq.) is reacted according to the general synthetic protocol with 4-nitroacetophenone (0.58 mmol, 96.6 mg, 1 eq.). The reaction mixture is stirred at 50° C. for 1 h. The hydrazone compound (1f) is obtained in the form of an orange-yellow powder (162 mg, 81%).

¹H RMN (CDCl₃, 500 MHz): δ 11.59 (s, 1H, N—H), 9.195 (s, 1H), 8.43 (dd, 1H), 8.315 (d, 2H), 8.142 (d, 1H), 8.02 (d, 2H), 2.518 (s, 3H)

### g) Precise Modalities for Preparing Compound 2a

The paragraph below illustrates the precise modalities for preparing compound 2a complying with the following formula:

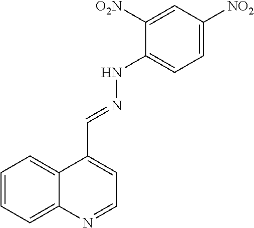 [SMILES: O=[N+]([O-])c1ccc(N/N=C/c2ccnc3ccccc23)c([N+](=O)[O-])c1]

2,4-dinitrophenylhydrazine (0.964 mmol, 281.2 mg, 1.3 eq.) is reacted according to the general procedure with 4-quinolinecarboxaldehyde (0.741 mmol, 120.1 mg, 1 eq.). The reaction mixture is stirred at 50° C. for 4 h. The hydrazone compound (2a) is obtained in the form of a yellow powder (255 mg, 100%).

### h) Precise Modalities for Preparing Compound 2b

The paragraph below illustrates the precise modalities for preparing compound 2b complying with the following formula:

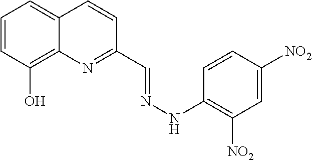 [SMILES: O=[N+]([O-])c1ccc(N/N=C/c2ccc3cccc(O)c3n2)c([N+](=O)[O-])c1]

2,4-dinitrophenylhydrazine (1.104 mmol, 322.1 mg, 1.3 eq.) is reacted according to the general procedure with 8-hydroxy-2-quinolinecarboxaldehyde (0.849 mmol, 151.5 mg, 1 eq.). The reaction mixture is stirred at 50° C. for 2 h. The hydrazone compound (2b) is obtained in the form of a yellow powder (234.56 mg, 78.2%).

### i) Precise Modalities for Preparing Compound 3a

The paragraph below illustrates the precise modalities for preparing compound 3a complying with the following formula:

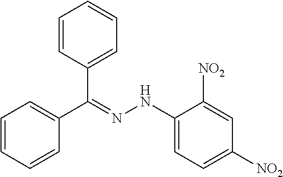 [SMILES: O=[N+]([O-])c1ccc(NN=C(c2ccccc2)c2ccccc2)c([N+](=O)[O-])c1]

2,4-dinitrophenylhydrazine (4.414 mmol, 1.21 g, 1.3 eq.) is reacted according to the general synthetic protocol with benzophenone (2.76 mmol, 0.508 g, 1 eq.). The reaction mixture is refluxed for 1 h and followed by CCM (toluene:THF 99:1, Rf=0.67).

The hydrazone compound (3a) is obtained in the form of an orange powder (0.98 g, 98%).

The IR and ¹H RMN spectroscopic results figure below.

IR: 3,302 (NH), 3,135 to 2,979 (Ar—H), 1,604 (C═N), 1,583, 1,406, 1,333, 1,281, 1,125, 1,062, and 698 cm−1

¹H RMN (DMSO-d₆, 400 MHz): δ (ppm) 11.25 (1H, s, N—H), 8.829 (s, 1H, ArDNPH-H), 8.472 to 8.2531 (dd, 2H, ArDNPH-H), 7.69 (m, 5H, Ar—H), 7.49 (s, 5H, Ar—H)

### j) Precise Modalities for Preparing Compound 3b

The paragraph below illustrates the precise modalities for preparing compound 3b complying with the following formula:

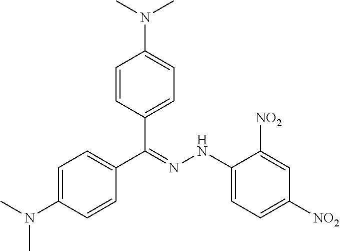 [SMILES: CN(C)c1ccc(C(=NNc2ccc([N+](=O)[O-])cc2[N+](=O)[O-])c2ccc(N(C)C)cc2)cc1]

2,4-dinitrophenylhydrazine (0.58 mmol, 169.2 mg, 1.3 eq.) is reacted in methanol according to the general synthetic protocol with 4,4′-bis(dimethylamino)-benzophenone (0.45 mmol, 120.9 mg, 1 eq.). The reaction mixture is refluxed for 4 h and followed by CCM (toluene:THF 98:2, Rf=0.3). The hydrazone compound (3b) is obtained in the form of a very dark purple powder (162.8 mg, 81.4%).

The IR and ¹H RMN spectroscopic results figure below.

IR: 3,275 (NH), 3,000 to 2,800 (Ar—H), 1,607 (C═N), 1,590, 1,512, 1,328, 1,132, 1,083, 827 and 741 cm⁻¹

¹H RMN (DMSO-d₆, 400 MHz): δ (ppm) 11.29 (s, 1H, N—H), 8.81 (s, 1H), 8.35 (d, 1H), 8.12 (d, 1H), 7.36 (dd, 2H), 7.05 (dd, 2H), 6.78 (dd, 2H), 6.59 (dd, 2H)

### k) Precise Modalities for Preparing Compound 3c

The paragraph below illustrates the precise modalities for preparing compound 3c complying with the following formula:

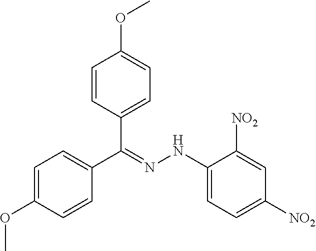 [SMILES: COc1ccc(C(=NNc2ccc([N+](=O)[O-])cc2[N+](=O)[O-])c2ccc(OC)cc2)cc1]

2,4-dinitrophenylhydrazine (0.58 mmol, 168.9 mg, 1.3 eq.), is reacted in methanol according to the general protocol with 4,4′-dimethoxybenzophenone (0.45 mmol, 110.6 mg, 1 eq.). The reaction mixture is refluxed for 1 h and followed by CCM (Cyclohexane:AcOEt 70:30, Rf=0.62). The hydrazone compound (3c) is obtained in the form of a slightly sparkly red powder (162 mg, 81%).

The IR and ¹H RMN spectroscopic results figure below.

IR: 3,273 (NH), 3,000 to 2,800 (Ar—H), 1,614 (C═N), 1,587, 1,503, 1,334, 1,251, 1,136, 1,025, 1,083 and 840 cm−1

¹H RMN (DMSO-d₆, 400 MHz): δ (ppm) 11.56 (s, 1H, N—H), 8.828 (s, 1H, ArDNPH-H), 8.435 (dd, 1H, ArDNPH-H), 8.21 (d, 1H, ArDNPH-H), 7.59 (d, 2H, Ar—H), 7.405 (d, 2H, Ar—H), 7.24 (d, 2H, Ar—H), 7.02 (d, 2H, Ar—H), 3.896 (s, 3H, OCH₃), 3.818 (s, 3H, OCH₃)

### l) Precise Modalities for Preparing Compound 4

The paragraph below illustrates the precise modalities for preparing compound 4 complying with the following formula:

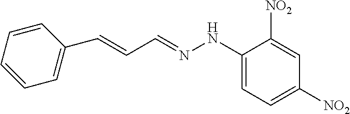 [SMILES: O=[N+]([O-])c1ccc(N/N=C/C=C/c2ccccc2)c([N+](=O)[O-])c1]

Trans-cinnamaldehyde (0.32 mmol, 0.041 mL, 1 eq.) in liquid form is directly added while hot on 2,4-dinitrophenylhydrazine (0.416 mmol, 121.5 mg, 1,3 eq.). The reaction mixture is stirred at 50° C. for 15 min. The hydrazone (1c) is obtained in the form of a red color powder (88.5 mg, 88.5%).

The IR, ¹H RMN and ¹³C RMN spectroscopic results figure below.

IR: 3,302 (NH), 3,135 to 2,979 (Ar—H), 1,604 (C═N), 1,583, 1,406, 1,333, 1,281, 1,125, 1,062, and 698 cm⁻¹

¹H RMN (CDCl₃, 400 MHz): δ (ppm) 11.5 (s, 1H, N—H), 9.0143 (s, 1H, ArDNPH-H), 8.356 to 8.326 (dd, 1H, ArDNPH-H), 8.006 to 7.982 (d, 1H, ArDNPH-H), 7.946 (d, 1H, N═CH), 7.52 (d, 2H), 7.425 to 7.36 (m, 3H, Ar—H), 7.03 (m, 2H, C═CH)

¹³C RMN (CDCl₃, 400 MHz): δ (ppm) 149.84 (HC═N), 141.18 (CAr—NHN), 130.04, 129.49, 128.86, 127.33, 124.07, 123.51, 116.77

### m) Precise Modalities for Preparing Compound 5a

The paragraph below illustrates the precise modalities for preparing compound 5a complying with the following formula:

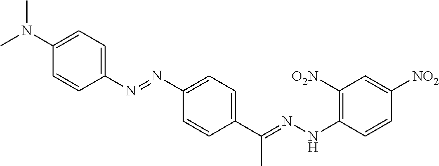 [SMILES: C/C(=N\Nc1ccc([N+](=O)[O-])cc1[N+](=O)[O-])c1ccc(/N=N/c2ccc(N(C)C)cc2)cc1]

2,4-dinitrophenylhydrazine (0.97 mmol, 257 mg, 1.3 eq.), is reacted according to the general procedure with 4-(4-dimethylamino)phenylazo)acetophenone (0.75 mmol, 200 mg, 1 eq.) in the presence of 4 equivalents of sulphuric acid. The reaction mixture is refluxed for 2 h. The hydrazone compound (5a) is obtained in the form of a brick red powder (267.7 mg, 80%).

### n) Precise Modalities for Preparing Compound 5b

The paragraph below illustrates the precise modalities for preparing compound 5b complying with the following formula:

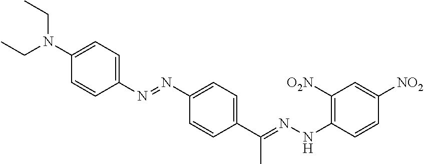 [SMILES: CCN(CC)c1ccc(/N=N/c2ccc(/C(C)=N/Nc3ccc([N+](=O)[O-])cc3[N+](=O)[O-])cc2)cc1]

2,4-dinitrophenylhydrazine (0.04 mmol, 12.9 mg, 1.3 eq.), is reacted according to the general procedure with 4-(4-diethylamino)phenylazo)acetophenone (0.03 mmol, 10 mg, 1 eq.) solubilised at 0.025 M in ethanol. The reaction mixture is refluxed for 2 h. The hydrazone compound (5b) is obtained in the form of a brick red powder (12.3 mg, 76.4%).

### o) Precise Modalities for Preparing Compound 6

The paragraph below illustrates the precise modalities for preparing compound 6 complying with the following formula:

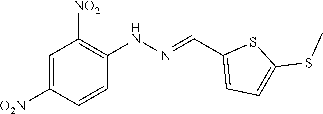 [SMILES: CSc1ccc(/C=N/Nc2ccc([N+](=O)[O-])cc2[N+](=O)[O-])s1]

2,4-dinitrophenylhydrazine (0.769 mmol, 224.5 mg, 1.3 eq.), is reacted according to the general procedure with 5-(methylthio)thiophene-2-carbaldehyde (0.592 mmol, 96.4 mg, 1 eq.). The reaction mixture is stirred at 50° C. for 2 h30. The hydrazone compound (6) is obtained in the form of a brick red powder (175.14 mg, 87.6%).

The ¹H RMN spectroscopic results figure below.

¹H RMN (CDCl₃, 400 MHz): δ 11.26 (s, 1H, N—H), 9.137 (d, 1H), 8.3314 to 8.3615 (dd, 1H), 8.1696 (s, 1H), 7.997 to 7.973 (d, 1H), 7.189 (d, 1H), 6.959 (d, 1H), 2.608 (s, 3H)

### p) Precise Modalities for Preparing Compound 7

The paragraph below illustrates the precise modalities for preparing compound 7 complying with the following formula:

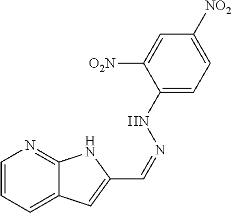 [SMILES: O=[N+]([O-])c1ccc(N/N=C\c2cc3cccnc3[nH]2)c([N+](=O)[O-])c1]

2,4-dinitrophenylhydrazine (0.797 mmol, 231.7 mg, 1.3 eq.), is reacted according to the general procedure with 7-azaindole-3-carboxaldehyde (0.613 mmol, 92.4 mg, 1 eq.). The reaction mixture is stirred at 50° C. for 15 minutes. The hydrazone compound (7) is obtained in the form of a brick red powder (152.78 mg, 76.4%).

The 1H RMN spectroscopic results figure below.

¹H RMN (DMSO-d₆, 400 MHz): δ 12.311 (s, 1H, N—H), 11.632 (s, 1H, N—H), 8.882 (s, 1H), 8.338 (s, 1H), 8.585 (d, 1H), 8.385 (m, 2H), 8.08 (s, 1H), 8.06 (s, 1H), 7.30 (m, 1H).

### Example 2

In this example, compounds 1d, 1f, 3c, 1b, 2a, 2b and 6 of which the preparation is described in Example 1 above are deposited respectively, via a solution comprising the given compound (10 mM) in dimethyl sulfoxide, on distinct supports based on fibreglass. The supports are subsequently dried naturally in order to evaporate the dimethyl sulfoxide then scanned to constitute test specimens before exposure to chemical compounds.

Each of the supports is subsequently subjected to 13 toxic compounds each deposited, at the rate of 1.6 L via a multi-channel electronic pipette, into individual cells provided on the support. Each of the supports is subsequently scanned after 5 minutes then after 1 hour of exposure to the toxic compounds.

The 13 toxic compounds are G-serie organophosphorus compounds (Sarin, Soman and Tabun (GA, CAS no. 77-81-6), a V-serie organophosphorus compound (the VX), vesicants (sulphur mustard (known as HD, CAS no. 505-60-2), nitrogen mustard (known as HN-3, CAS no. 555-71-1), lewisite (known as L1, CAS no. 541-25-3), arsenic compounds (diphenylchlorarsine known as “C1” and diphenylcyanoarsine known as “C2”) and the organophosphorus compounds complying with the following formulae (XXVII), (XXVIII), (XXIX) and (XXX):

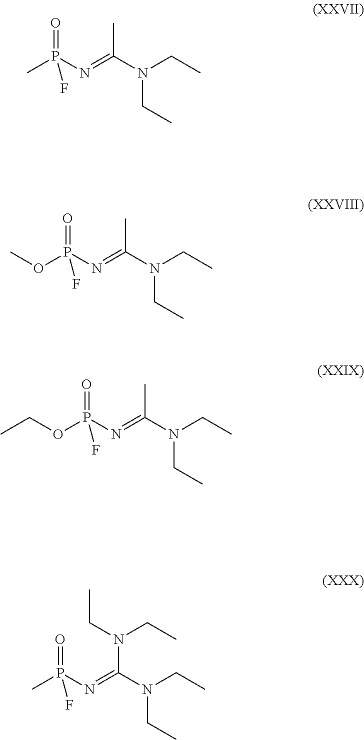

For each of the supports, the exposure to the organophosphorus compounds of formulae (XXVII), (XXVIII), (XXIX) and (XXX) generates a color change from yellow or orange to purple, blue or brown and this instantaneously, whereas there is no change for the other compounds.

Thus, this example certifies the specificity of detecting organophosphorus compounds of formulae (XXVII), (XXVIII), (XXIX) and (XXX) by the hydrazone compounds mentioned above.

### Example 3

In this example, compounds 1f, 1b, 3a, 5a, of which the preparation is described in Example 1 above, and the compounds of the following formulae (V) and (XI):

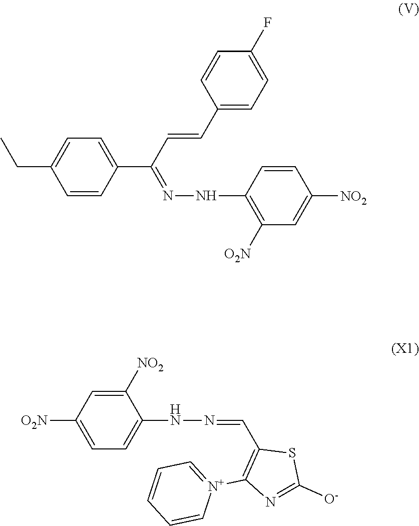 [SMILES: CCc1ccc(C(/C=C/c2ccc(F)cc2)=N/Nc2ccc([N+](=O)[O-])cc2[N+](=O)[O-])cc1 ; O=[N+]([O-])c1ccc(NN=Cc2sc([O-])nc2-[n+]2ccccc2)c([N+](=O)[O-])c1]

are respectively deposited, via a solution comprising the given compound (10 mM) in dimethyl sulfoxide, on distinct supports based on fibreglass. The supports are subsequently dried naturally in order to evaporate the dimethyl sulfoxide then scanned to constitute test specimens before exposure to chemical compounds.

Each of the supports is subsequently subjected to 4 toxic compounds each deposited, at the rate of 1.6 μL via a multi-channel electronic pipette, into individual cells provided on the support. Each of the supports is subsequently scanned after 5 minutes then after 1 hour of exposure to the toxic compounds.

The 4 toxic compounds comply with the following formulae (XXVII), (XXVIII), (XXIX) and (XXX):

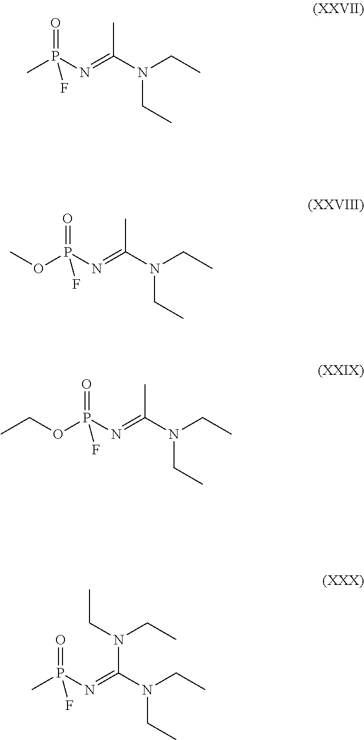

Placing the aforementioned 4 organophosphorus neurotoxic agents in contact with the supports made of fibreglass, whereon the hydrazone compounds mentioned above were deposited, results in a color change thereof, thus certifying the efficiency of the supports in accordance with the invention, for detecting this specific type of organophosphorus compounds.

### Example 4

In this example, compounds 1a, 1b, 1c, 1d, 1e, 1f, 2a, 2b, 3a, 3b, 3c, 4, 5a, 5b and 6 of which the preparation is described in Example 1 above, the compounds of formulae (V) and (XI) as defined above and the compounds of the following formulae:

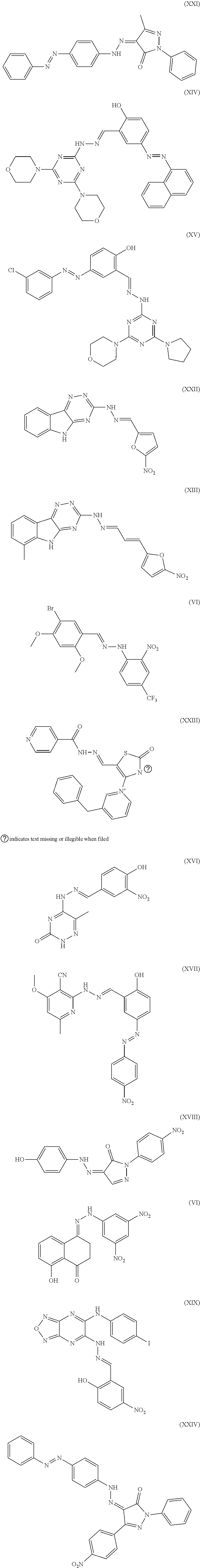

are respectively deposited, via a solution comprising the given compound (10 mM) in dimethyl sulfoxide, on distinct supports based on fibreglass. The supports are subsequently dried naturally in order to evaporate the dimethyl sulfoxide then scanned to constitute test specimens before exposure to chemical compounds.

Each of the supports is subsequently subjected to 4 toxic compounds each deposited, at the rate of 1.6 μL via a multi-channel electronic pipette, into individual cells provided on the support. Each of the supports is subsequently scanned after 5 minutes then after 1 hour of exposure to the toxic compounds. The 4 toxic compounds are those defined in Example 3 below.

Placing the aforementioned 4 organophosphorus neurotoxic agents in contact with the supports made of fibreglass, whereon the hydrazone compounds mentioned above were deposited, results in a color change thereof, thus certifying the efficiency of the supports in accordance with the invention, for detecting this specific type of organophosphorus compounds.

### Example 5

In this example, compounds 1a, 1c, 1e, 1f, 2a, 2b, 3b, 4, of which the preparation is described in Example 1 above, the compounds of formulae (XXI), (VI), (XIX) of which the formulae are defined in Example 4 above and the compound of the following formula (XX):

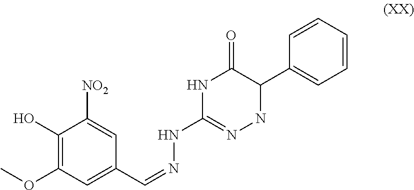 [SMILES: COc1cc(/C=N\NC2=NNC(c3ccccc3)C(=O)N2)cc([N+](=O)[O-])c1O]

are deposited respectively on distinct supports made of chromatography paper, said supports being subjected to four organophosphorus neurotoxic agents as defined in Example 3 above.

Placing the aforementioned 4 organophosphorus neurotoxic agents in contact with the supports made of chromatography paper, whereon the hydrazone compounds mentioned above were deposited, results in a color change thereof, thus certifying the efficiency of the supports in accordance with the invention, for detecting this specific type of organophosphorus compounds.

1. A colorimetric detection system usable for detecting a chemical compound, the system comprising:
a support comprising a color indicator of the chemical compound to be detected,
wherein the color indicator is a conjugated hydrazone compound of formula

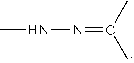

2. The system of claim 1, wherein the conjugated hydrazone compound comprises
a cyclic group bonded to the carbon atom of the hydrazone function and
an aromatic group bonded to the nitrogen atom located in a β-position to the carbon atom of the hydrazone group, the aromatic group comprising an electron-withdrawing group.

3. The system of claim 2, wherein the cyclic group bonded to the carbon atom of the hydrazone group is carbon cyclic group and the electron-withdrawing group of the aromatic group is NO₂.

4. The system of claim 1, wherein the conjugated hydrazone compound has (II), (III), (IV), (V), (VI), or (VI′): 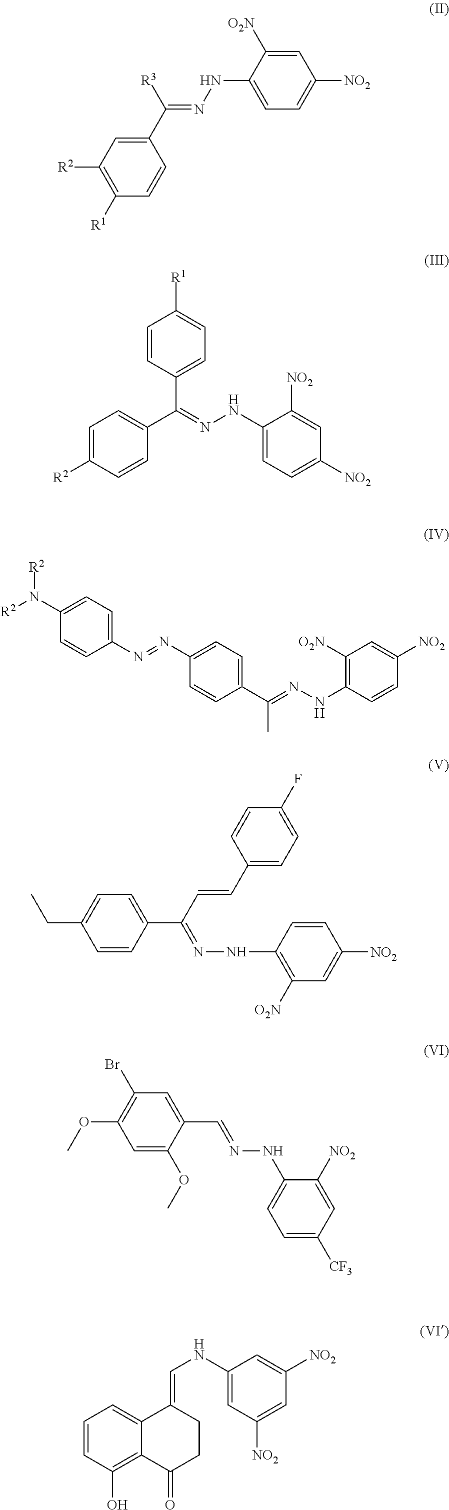
wherein
in formula (II), R¹ is N(CH₃)₂, CH₃, OCH₃, OH, SCH₃, or NO, R³ is H or OCH R³ is H or CH₃,
in formula (III), R¹ and R² are independently H, N(CH₃)₂, OCH₃, or NO₂,
in formula (IV), R¹ and R² are independently CH₃, or CH₂—CH₃.

5. The system of claim 4, wherein the conjugated hydrazone compound is the compound of formula (II) wherein
R¹ is N(CH₃)₂, R² is H, and R³ is H,
R¹ is CH₃, R² is H, and R³ is H,
R¹ is OCH₃, R² is H, and R³ is H,
R¹ is OH, R² is OCH₃, and R³ is H,
R¹ is SCH₃, R² is H, and R³ is H, or
R¹ is NO₂, R² is H, and R³ is CH₃.

6. The system of claim 4, wherein the conjugated hydrazone compound is the compound of formula (III) wherein
R¹ is H and R² is H,
R¹ is N(CH₃)₂ and R² is N(CH₃)₂, or
R¹ is OCH₃ and R² is OCH₃.

7. The system of claim 4, wherein the conjugated hydrazone compound is the compound of formula (IV) wherein
R¹ is CH₃ and R² is CH₃,
R¹ is CH₂—CH₃ and R² is CH₂—CH₃.

8. The system of claim 2, wherein the cyclic group bonded to the carbon atom of the hydrazone function is a heteroaromatic group and the electron-withdrawing group of the aromatic group is NO₂.

9. The colorimetric detection system of claim 8, wherein the conjugated hydrazone compound is of formula (VII), (VIII), (IX), (X), or (XI): 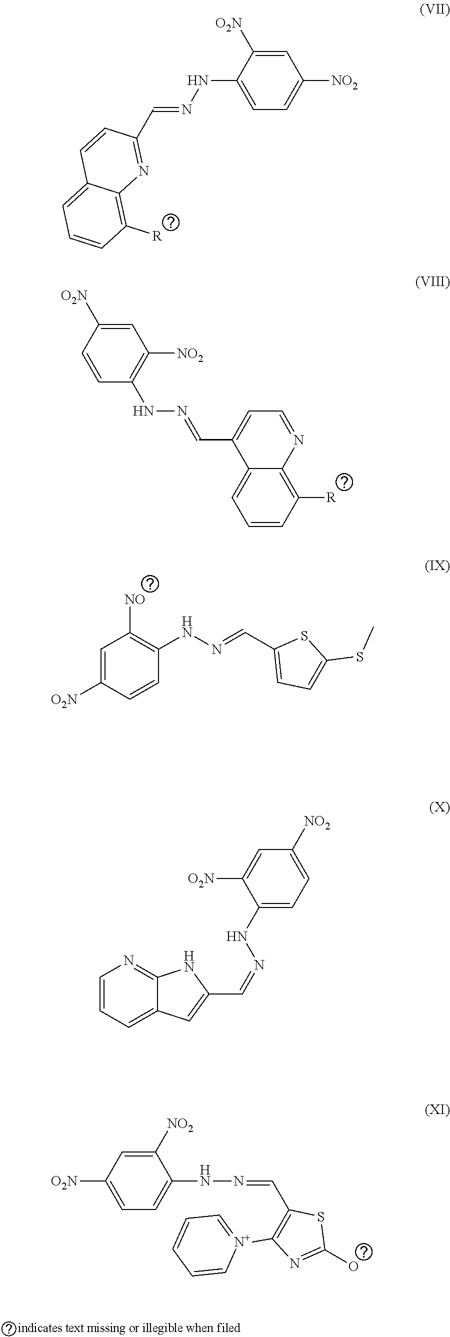
wherein:
in formula (VII), R¹ is H or OH;
in formula (VIII), R¹ is H or OH.

10. The system of claim 1, wherein the conjugated hydrazone compound comprises
an ethylenic group bonded to the carbon atom of the hydrazone group and
a second aromatic group comprising a second electron-withdrawing group.

11. The system of claim 10, wherein the conjugated hydrazone compound is of formula (XII) or (XIII):

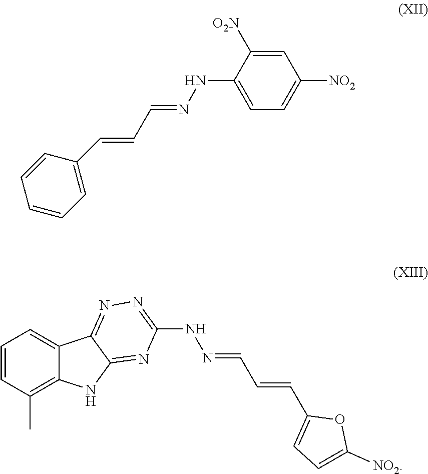 [SMILES: O=[N+]([O-])c1ccc(N/N=C/C=C/c2ccccc2)c([N+](=O)[O-])c1 ; Cc1cccc2c1[nH]c1nc(N/N=C/C=C/c3ccc([N+](=O)[O-])o3)nnc12]

12. The system of claim 1, wherein the conjugated hydrazone compound comprises an aromatic group bonded to the nitrogen atom in the β-position to the carbon atom of the hydrazone group or bonded to the carbon atom of the hydrazone group,
wherein the aromatic group comprises an OH group.

13. The colorimetric detection system of claim 12, wherein the conjugated hydrazone compound is of formula (XIV), (XV), (XVI), (XVII), (XVIII), (XIX), or and (XX):

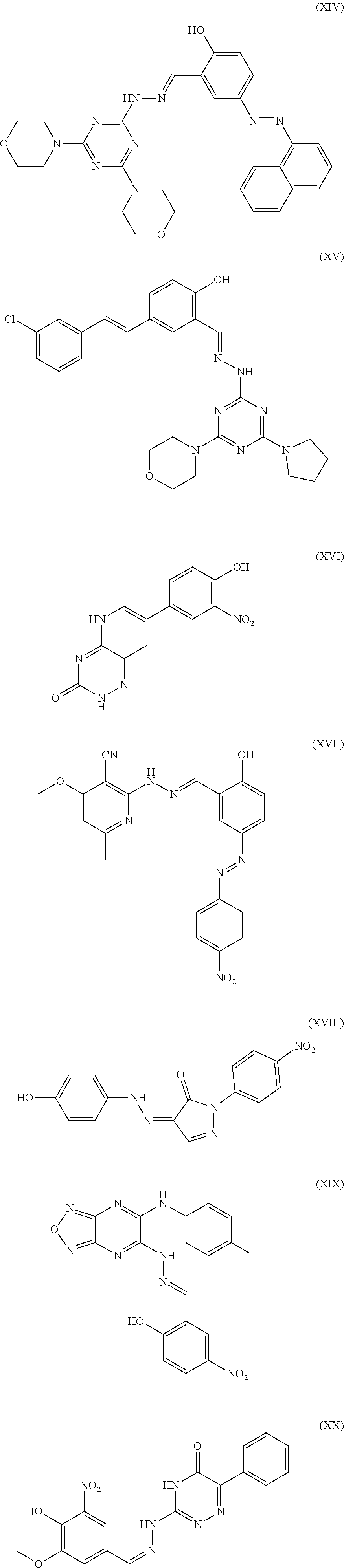

14. The system of claim 1, wherein the conjugated hydrazone compound comprises (i) a first heteroaromatic group bonded to the nitrogen atom in the β-position to the carbon atom of the hydrazone group and (ii) a second heteroaromatic group bonded to the carbon atom of the hydrazone group, or
wherein the conjugated hydrazone compound comprises a diazoaromatic group bonded to the nitrogen atom in the β-position to the carbon atom of the hydrazone group and a heteroaromatic group encompassing the carbon atom of the hydrazone group.

15. The system of claim 14, wherein the conjugated hydrazone compound is of formula (XXI), (XXII), (XXIII), or (XXIV):

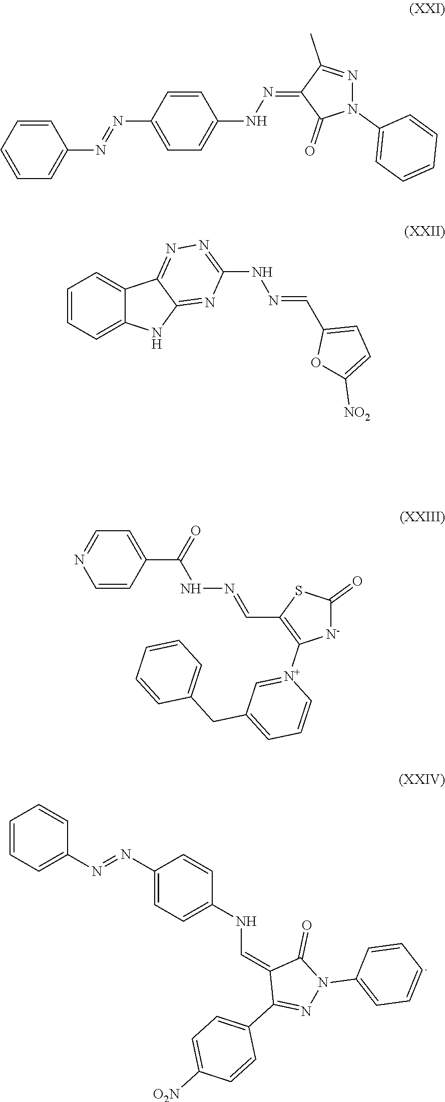 [SMILES: CC1=NN(c2ccccc2)C(=O)/C1=N\Nc1ccc(/N=N/c2ccccc2)cc1 ; O=[N+]([O-])c1ccc(/C=N/Nc2nnc3c(n2)[nH]c2ccccc23)o1 ; O=C(N/N=C/c1sc(=O)[n-]c1-[n+]1cccc(Cc2ccccc2)c1)c1ccncc1 ; O=C1/C(=C\Nc2ccc(/N=N/c3ccccc3)cc2)C(c2ccc([N+](=O)[O-])cc2)=NN1c1ccccc1]

16. The system of claim 1, wherein the support comprises paper.

17. The system of claim 1, wherein the support comprises fiberglass.

18. A hydrazone compound having formula (5a), (5b), or (X):

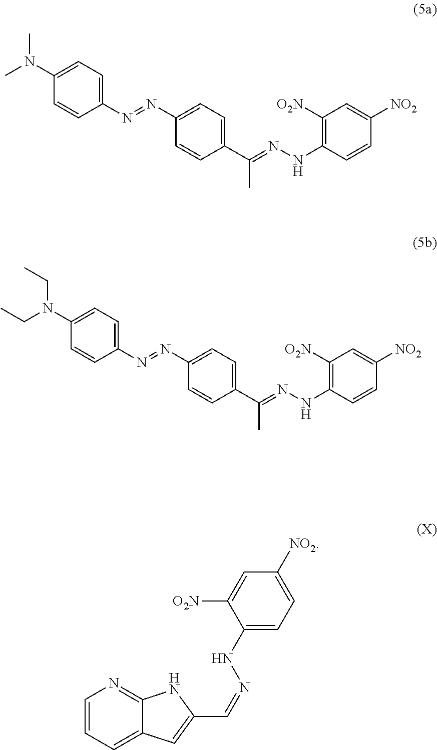 [SMILES: C/C(=N\Nc1ccc([N+](=O)[O-])cc1[N+](=O)[O-])c1ccc(/N=N/c2ccc(N(C)C)cc2)cc1 ; CCN(CC)c1ccc(/N=N/c2ccc(/C(C)=N/Nc3ccc([N+](=O)[O-])cc3[N+](=O)[O-])cc2)cc1 ; O=[N+]([O-])c1ccc(N/N=C\c2cc3cccnc3[nH]2)c([N+](=O)[O-])c1]

19. A method for detecting a chemical compound in a medium, the method comprising:
placing the medium in contact with the colorimetric detection system of claim 1;
deducing presence or absence of the chemical compound in the medium, depending on a possible chromatic change of the color indicator of the colorimetric detection system.

20. The method of claim 19, wherein the chemical compound comprises a toxic warfare compound, toxic industrial compound, and/or pesticide.

21. The method of claim 20, wherein the chemical compound has formula (XXV) or (XXVI): 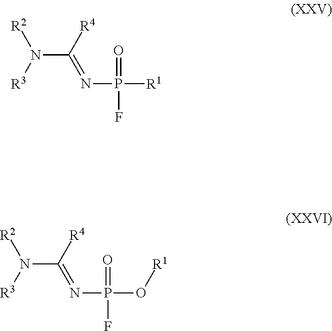
wherein:
R¹ is H or an alkyl or cycloalkyl group comprising at most 10 carbon atoms;
R² and R³ are independently an alkyl or cycloalkyl group comprising at most 10 carbon atoms, and
R⁴ is H, an amino group, or an alkyl or cycloalkyl group comprising at most 10 carbon atoms.

22. A colorimetric detection kit suitable for detecting a chemical compound, the kit comprising:
the colorimetric detection system of claim 1; and
a colorimetric scale configured for making a correspondence between a chromatic change observed and the chemical compound detected.
